(19) United States
(12) Patent Application Publication
(10) Pub. No.: US 2005/0214248 A1
(43) Pub. Date: Sep. 29, 2005

(54) Non-glycosylated polyacrylamide conjugates and their use for cytoprotection

(75) Inventors: Robert Rieben, Hinterkappelen (CH); Paul Mohacsi, Herrliberg (CH); Nicolai Vladimirovich Bovin, Moscow (RU); Elena Yuriovna Korchagina, Moscow (RU)

Correspondence Address: JOYCE VON NATZMER, 4615 NORTH PARK AVENUE, SUITE 919, CHEVY CHASE, MD, 20815, US

(73) Assignees: University of Bern; Shemyakin and Ovchinnikov Institute Institute of Bioorganic Chemistry

(21) Appl. No.: 10/524,357

(22) PCT Filed: Aug. 12, 2003
(86) PCT No.: PCT/EP03/08987

§ 371 Date: Feb. 14, 2005

(30) Foreign Application Priority Data
Aug. 16, 2002 (EP) 02018495.8

(51) Int. Cl. A61K 31/785; C08F 265/02

(52) U.S. Cl. 424/78.27; 525/301

(57) ABSTRACT

The present invention relates to new non-glycosylated polyacrylamide conjugates, a method for protecting endothelial cells from complement-mediated cellular damage and the use of said non-glycosylated polyacrylamide conjugates as a medicament.

### FIELD OF THE INVENTION

The present invention relates to new non-glycosylated polyacrylamide conjugates, a method for protecting endothelial cells from complement-mediated cytotoxicity and the use of said non-glycosylated polyacrylamide conjugates as a medicament.

### BACKGROUND OF THE INVENTION

Complement mediated damage of—mainly endothelial—cells plays an important role in a number of different pathophysiological processes, among which are acute vascular rejections of allo- and xenografts, ischemia/reperfusion injury (I/R injury), severe sepsis or septic shock, and arteriosclerosis. One example from the clinical practice is organ transplantation, in which no satisfactory therapeutic options are available to prevent and/or treat the I/R injury of the graft, nor to treat acute vascular rejection reactions that may occur due to preformed antibodies. In both cases complement-mediated damage to mainly endothelial cells plays a major role. In the case of HLA-immunized recipients or in ABO-incompatible- and xenotransplantation, specific antibodies bind to the graft endothelium and are responsible for activation of the complement system and subsequently the endothelial cells themselves. In I/R injury the ischemia during storage of the graft leads to changes on the endothelium that render it susceptible to complement- and coagulation-mediated damage upon reperfusion. The use of soluble complement inhibitors/endothelial cell protectants might therefore be a valid therapeutical approach for both clinical problems.

To date, depletion of the central complement component C3 by cobra venom factor is the most efficient experimental treatment, but results in the loss of major defense mechanisms against microbial pathogens. Therefore, it has been the aim of many researchers in this area to develop selective complement inhibitors.

Another approach to prevent endothelial damage and inflammation is the use of selectin inhibitors. Selectins are involved in the cooperative multistep process of leukocyte trafficking from blood vessels into sites of inflammation (Rosen, S. D., and Bertozzi, C. R., (1994). The selectins and their ligands. Curr. Opin. Cell Biol 6, 663-673). The selectins share the ability to recognize the tetrasaccharide SiaLe^{x} (sialyl Lewis X). The binding affinities of monomeric SiaLe^{x} and its mimetics are poor, being in the millimolar range. However, multimeric negatively charged molecules such as heparin, inositol hexaphosphate, sulfatide, and especially the polysaccharide fucoidan are known to be P-selectin inhibitors (Skinner et al. (1989) Characterization of human platelet GMP-140 as a heparin-binding protein. Biochem.-Biophys. Res. Communs 164, 1373-1379; Marinier et al. Sulfated galactocerebro-sides as potential antiinflammatory agents. J. Med. Chem., 1997, 40, 3234-3247; Cecconi et al. Inositol polyanions. Noncarbohydrate inhibitors of L- and P-selectin that block inflammation. J. Bio. Chem., 269,15060-15666 (1994)).

Ushakova et al. (Vopr. Med. Khim, 45:5, 375-383, 2000) describes an inhibitory activity of monomeric and polymeric selectin ligands. According to these authors (SiaLe^{x}, SiaLe^{a}, and HSO₃Le^{x}) tetrasaccharides, their conjugates with polyacrylamide and a number of further substances block selectins similar to the polysaccharide fucoidan. PAA conjugates containing the ligand tyrosine-O-sulfate in addition to the above mentioned oligosaccharides were the most potent of the tested synthetic selectin blockers. In comparison to the most potent known inhibitor fucoidan, the biligand glycoconjugate HSO₃Le^{a}-PA-sTyr displayed similar activity in blocking L-selectin, while its activity towards P-selectin was 10 times lower. All tested synthetic polymers were able to inhibit neutrophil extravasation to inflammation sites in a concentration of about 10 mg/kg.

The manufacturing of glycosylated, in particular sialylated, PAA conjugates is very expensive. The cost of producing a substance such as SiaLe^{x}-PAA lies around 10.000 EUR/100 mg.

Therefore, there is a need for cost effective substances that selectively inhibit cellular, in particular complement-mediated endothelial cell damage mediated by the patient's natural immunity, such as the complement system, without having systemic effects on blood coagulation.

In particular, there is a need for treating and/or preventing inflammatory reactions towards endothelial cells, especially in a number of diseases such as arteriosclerosis, chronic heart failure, cardiac or brain infarction, surgery related ischemia, vascular rejection reactions, and sepsis, acute respiratory distress syndrome (ARDS), septic shock. Similarly, reactions mediated by the innate immune system (comprising the complement, coagulation, and kinin systems, natural antibodies, phagocytic, dendritic and NK cells) are involved in the pathogenesis of allogeneic or xenogeneic islet cells rejection as well as in HIV infection.

### DISCLOSURE OF THE INVENTION

One aspect of the present invention provides new polyacrylamide conjugates of the general formula I, 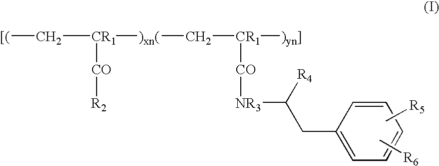
wherein
R₁ denotes hydrogen or methyl,
R₂ denotes N(R₇R₈) or OH,
R₃ denotes a hydrogen, C₁₋₆ alkyl or C₃₋₆ cycloalkyl,
R₄ denotes H or COO⁻M⁺,
R₅, R₆ denote, in each case independently of one another a hydrogen, SO₃⁻M⁺ or OSO₃⁻M⁺,
R₇, R₈ denote, in each case independently of one another, hydrogen, C₁₋₁₆ alcohol, C₁₋₆ alkyl, phenyl, benzyl, phenethyl or N(R₇R₈) denotes a N(CH₂)₂₋₆ ring that may also be substituted,
n is 20 to 500,
y is from 0.2 to 1.0,
x is 1-y,
M⁺ is a physiologically acceptable monovalent cation.
   and their diastereomers or enantomers in the form of their acids or salts of physiologically compatible bases.

Preferably, in said polyacrylamide conjugate R₁ denotes hydrogen.

In a preferred embodiment said polyacrylamide conjugate comprises a substituent R₂ which denotes N(R₇R₈).

Furthermore, it is also preferred that the substituent R₃ in the above formula denotes hydrogen.

In a preferred embodiment, the present invention relates to polyacrylamide conjugates, wherein R₄ denotes COO⁻M⁺.

In a further preferred embodiment, the present invention relates to polyacrylamide conjugates, wherein R₆ is hydrogen and R₅ is SO₃⁻M⁺ or OSO₃⁻M⁺ in the meta or para position, preferably in the para position, most preferably R₅ is OSO₃⁻M⁺ in the para position.

In a more preferred embodiment R₅ and R₆ both denote hydrogen.

Preferably, R₇ is hydrogen and R₈ is a C₁₋₆ alcohol, more preferably a C₁₋₄ alcohol, most preferably ethyl alcohol.

Polyacrylamide conjugates according to the invention comprise a counterion M⁺, which may be any suitable pharmaceutically acceptable cation, preferably selected from the group of Na⁺, K⁺, NH₄⁺, Et₃NH⁺, HO(CH₂)₂NH₃⁺.

Polyacrylamide conjugates according to the invention preferably comprise 20 to 400 of the units (n) shown in the above formula. More preferably n is 20 to 300, or even 20 to 100, and most preferably about 20 to 80.

The ratio of the components x and y in the above formula is preferably such that y is 0.2 to 0.8, more preferably 0.3 to 0.6 or even 0.3 to 0.5, most preferably 0.35 to 0.45.

The SiaLe^{x}/SiaLe^{a}-free polyacrylamide conjugates bearing highly dense sulfated tyrosine or tyramine according to the present invention are excellent site specific blockers of complement activation and P-selectin mediated leucocyte adhesion in vitro and in vivo, being even more active in vitro than fucoidan or low molecular weight dextran sulfate (DXS), the most potent known inhibitors up to now. They interfere much less with blood coagulation than the latter substances. They protect endothelial cells from complement-mediated cytotoxicity. Although the present invention is by no means bound by theory, it seems that that endothelial cell protection of these substances is due to the at least functional replacement of the natural heparan sulfate proteoglycans that are shed from the cell surface upon damage-induced activation of the endothelial cells.

The polyacrylamide conjugates according to the present invention are excellent compounds of choice for inhibiting complement activation and P-selectin mediated leukocyte adhesion in vitro. They are preferably employed in basic scientific research as well as in the development of new medicaments and therapies for diseases in which the innate immune system, including complement, plays a major pathophysiological role.

Furthermore, the polyacrylamide conjugates according to the present invention are excellent compounds of choice for protecting endothelial cells from complement-mediated cytotoxicity. In a preferred embodiment the present invention relates to a method for protecting endothelial cells from complement-mediated cytotoxicity comprising the addition of at least one polyacrylamide conjugate according to the present invention to said cells in vitro.

The compounds of the present invention have also demonstrated their efficacy in vivo, Therefore, polyacrylarnide conjugates according to the invention are useful as a medicament. Preferably, said compounds are used for the preparation of a medicament for protecting endothelial cells from complement-mediated damage.

Chronic heart failure and arteriosclerosis are diseases that are closely linked to complement-mediated endothelial damage and dysfunction. (Gullestad et al. Circulation, Jan. 16, 2001, 220-224, Aukrust et al. Circulation, Sep. 25, 2001, 1494-1500, Buono et al. Circulation, Jun. 25, 2002, 3025-3031) The compounds of the present invention are preferably used for the preparation of a medicament for the prevention and/or treatment of inflammatory reactions towards endothelial cells, preferably in arteriosclerosis or chronic heart failure.

In effecting treatment of a subject suffering from diseases associated with complement-mediated damage the compounds disclosed by the present invention for said purpose can be administered in any form or mode which makes the therapeutic compound bioavailable in an effective amount, including oral or parenteral routes. For example, products of the present invention can be administered intraperitoneally, intranasally, buccally, topically, orally, subcutaneously, intramuscularly, intravenously, transdermally, rectally, and the like. One skilled in the art of preparing formulations can readily select the proper form and mode of administration dependingzuponthe-particular characteristics of the product selected, the disease or condition to be treated, the stage of the disease or condition, and other relevant circumstances. (Remington's Pharmaceutical Sciences, Mack Publishing Co. (1990)). The products of the present invention can be administered alone or in the form of a pharmaceutical preparation in combination with pharmaceutically acceptable carriers or excipients, the proportion and nature of which are determined by the solubility and chemical properties of the product selected, the chosen route of administration, and standard pharmaceutical practice. For oral application suitable preparations are in the form of tablets, pills, capsules, powders, lozenges, sachets, cachets, suspensions, emulsions, solutions, drops, juices, syrups, while for parenteral, topical and inhalative application suitable forms are solutions, suspensions, easily reconstitutable dry preparations as well as sprays. Compounds according to the invention in a sustained-release substance, in dissolved form or in a plaster, optionally with the addition of agents promoting penetration of the skin, are suitable percutaneous application preparations. The products of the present invention, while effective themselves, may be formulated and administered in the form of their pharmaceutically acceptable salts, such as acid addition salts or base addition salts, for purposes of stability, modulation of hydrophobicity, increased solubility, and the like.

The amount of active agent to be administered to the patient depends on the patient's weight, on the type of application, symptoms and the severity of the illness. Normally, 0.1 mg/kg to 25 mg/kg of at least one substance of the general formula I is administered, but when applied locally, e.g. intracoronary administration, much lower total doses are also possible.

In a preferred embodiment, the compounds of the present invention are useful for the preparation of a medicament for preventing ischemia/reperfusion damage.

In another preferred embodiment, the present invention relates to the use of said compounds for the preparation of a medicament for the treatment of cardiac or brain infarction.

Preferably, polyacrylamide conjugates according to the invention are used for the preparation of a medicament for preventing damage to organs during surgery-related ischemia, more preferably for the preparation of a medicament for preventing acute vascular rejection reactions.

More preferably, polyacrylamide conjugates according to the invention are used for the preparation of a medicament for preventing acute vascular rejection reactions in ABO-incompatible transplants or xenotransplants.

Due to its protective properties with respect to complement-mediated damage, the compounds of the present invention are particularly useful for the preparation of solutions and/or gels for safe-keeping of life donor organs for use in transplants.

More preferably, the compounds of the present invention are useful for the preparation of a medicament for use in allogeneic and xenogeneic islet transplantation.

Recently, sulfated polymers have entered clinical trials for the prevention and treatment of HIV such as Carregard, a seaweed extract, PRO 2000, a naphtalene sulfate polymer and dextrin 2 sulfate, all being polymers containing negatively charged molecules that block the electrostatic attraction between HIV's gp 120 envelope protein and target cells (Julia Clayton, BioMedNet News, May 16, 2002). In this context, although not bound by theory, the compounds of the present invention are deemed particularly useful for the preparation of a medicament for use in the prevention and/or treatment of HIV infection.

In another embodiment the present invention relates to the use of a polyacrylamide conjugate of the invention for the preparation of a medicament for use in the prevention and/or treatment of severe sepsis, acute respiratory distress syndrome (ARDS), or septic shock.

Abbreviations: SiaLe^{x}=sialyl Lewis X, Neu5Acα2-3Galβ1-4(Fucα1-3)GIcNAc; SiaLe^{a}=sialyl Lewis A, Neu5Acα2-3Galβ1-3(Fucα1-4)GIcNAc; HSO₃Le^{a}=3′-sulfo-Lewis A; 3′-HSO₃Galβ1-3(Fucα1-4)GIcNAc; sTyr=tyrosine-O-sulfate; PAA=polyacrylamide

### FIGURES

FIG. 1 demonstrates the inhibitory potency of five sTyr-PAA conjugates which differ in their sTyr loading in comparison to the polysaccharide fucoidan. The in vitro test-system is based on the inhibition of HSO₃Le^{a}-PAA-biotin binding to recombinant P-selectin. For details, see example 1.

FIG. 2 shows the effects of different PAA-conjugates as well as DXS (dextran sulfate) on complement activation in a standard hemolytic assay (CH50 test) with antibody-sensitized sheep erythrocytes.

Dose-response curves for the selected substances are provided in comparison to DXS. The sTyr-PAA conjugate with a 40% substitution rate was the best complement inhibitor of all substances tested, demonstrating a stronger inhibitory activity than DXS.

FIG. 3 shows the effect of DXS (dextran sulfate) as well as PAA-sTyr on complement and coagulation.

The figure demonstrates that DXS already leads to a prolongation of the aPTT (filled circles) to more than 300 seconds at. a concentration of 0.05 mg/ml with only a minimal inhibition of complement activation (empty circles) at that concentration. In contrast PAA-sTyr (40%) shows only a slight prolongation of the aPTT (filled squares) at 0.05 mg/ml, and reduces complement activation to 27% at that concentration (empty squares). Therefore, PAA-STyr with a 40% substitution rate was both, a better complement inhibitor than DXS and a much less potent anti-coagulant than the latter.

### EXAMPLES

The following examples further illustrate the best mode contemplated by the inventors for carrying out their invention.

### Example 1

### Inhibition of P-Selectin Mediated Leukocyte Adhesion and Extravasation

Reagents

All PAA-based neoglycoconjugates were from 30 to 40 kDa and obtained according to standard methods (Gordeeva, E. A., Tuzikov, A. B., Galanina, O. E., Pochechueva, T. V. & Bovin, N. V. (2000). Microscale synthesis of glycoconjugate series and libraries. Anal. Biochem. 278, 230-232; Bovin, N. V., Korchagina, E. Yu., Zemlyanukhina, T. V., Byramova, N. E., Galanina, O. E., Zemlyakov, A. E., Ivanov, A. E., Zubov, V. P., and Mochalova, L. V. (1993) Synthesis of polymeric neoglycoconjugates based on N-substituted polyacrylamide. Glycoconj. J. 10, 142-151). sTyr-PAA (containing 80 mol % sTyr) was synthesized using 200% excess of sTyr in relation to the activated polymer. The degree of sTyr substitution was determined by the conjugate's increase in weight. Recombinant ZZ-selectin (monovalent) lacking the transmembrane and cytosolic domains was produced as C-terminal chimera with the ZZ domain of protein A (Priest, R., Nawaz, S., Green, P. M., and Bird, M. I. (1995). Adhesion of eosinophils to E- & P-selectin. Biochem. Soc. Trans. 23, 162S). Tripeptide Tyr-Tyr-Tyr (Bachem, Germany) and aminoglucitol (Sigma) were per-O-sulfated with SO₃/Py complex (Field, R. A., Otter, A., Fu, W., Hindsgaul, O. Carbohydr. Res. 1995, 276, 347-363). Fucoidan was purchased from Sigma (USA).

In vitro Assay for Inhibition of P-Selectin Binding

The assay was performed on 96-Well plates. Human IgG was used as a primary coating reagent to immobilize recombinant selectin via the ZZ-domain of the fusion protein. The working concentration of selectin was 3 ng/well. HSO₃Le^{a}-PAA-biotin (Zemlyanukhina, T. V., Nifant'ev, N. E. , Shashkov, A. S., Tsvetkov, Y. E., and Bovin, N. V. (1995) Selectin receptors synthesis of spacer-armed sulfated trisaccharides Lewis A and Lewis X and neoglycoconjugates thereof. Carbohydrate Lett. 1, 277-284) was selected from HSO₃Le^{a}-, HSO₃Le^{x}-, SiaLe^{a}-, and SiaLe^{x}-PAA because it showed a maximum level of binding in this assay. Details of this assay were published in (Game, S. M., Rajapurohit, P. K., Clifford, M., Bird, M. I., Priest, R., Bovin, N. V., Nifant'ev, N. E., O'Beime, G., and Cook, N. D. (1998) Scintillation proximity assay for E-, P-, and L-selectin utilizing polyacrylamide-based neoglycoconjugates as ligands. Anal. Biochem. 258, 127-135; Tatyana V. Pochechueva, Oxana E. Galanina, Michael I. Bird, Nikolay E. Nifant'ev, Nicolai V. Bovin. Assembly of P-Selectin Ligands on a Polymeric Template, Chem&Biol, Vol. 9, 2002, p. 757-762; Natalia Ushakova, Marina Preobrazhenskaya, Mike I. Bird, Richard Priest, A. V. Semenov, A. V. Mazurov, Nikolay Ni-fant'ev, Tatyana Pochechueva, Oxaha Galanina, Nicolai Bovin. Monomeric and Multimeric Blockers of Selectins: Comparison of In Vitro and In Vivo Activity/Biol. Chem. Hoppe Seyler, 2002 submitted).

Rat Peritoneal Inflammation Model

Acute rat peritonitis was induced as described in (Preobrazhenskaya, M. E., Berman, A. E., Mikhailov, V. I., Ushakova, N. A., Mazurov, A. V., Semenov, A. V., Usov, A. I., Nifant'ev, N. E., and Bovin, N. V. (1997) Fucoidan inhibits leukocyte recruitment in a model peritoneal inflammation in rat and blocks interaction of P-selectin with its carbohydrate ligand. Biochem. Mol. Biol. Int 43, 443-451) with some modifications. 10 ml of PBS containing 10% peptone was injected intraperitoneally into female Wistar rats (180-220 g). After 3 h rats were sacrificed and their peritoneal cavities were laved with 30 ml PBS containing 60 units/ml of heparin, 0.02% EDTA and 0.03% bovine serum. The total cell number in the lavage was counted and the cell suspension was concentrated by centrifugation at 400 g for 10 min. After 1:1 dilution with bovine serum smears were prepared and stained according to the Pappenheim method. Neutrophils were counted on two parallel slides and the total neutrophil number was calculated. The inhibitors were administered intravenously as a single dose in 0.3 ml sterile 0.9% NaCl 15 min after peptone injection. The same volume of NaCl solution Was injected into control animals. A difference between the control and tested groups was analyzed for statistical significance based on Student's t-test, p-values<0.05 were considered to be significant.

Results of the in vitro P-Selectin Inhibition Assay

According to previous data on synthetic multivalent inhibitors, (e.g. Tatyana V. Pbchechueva, Oxana E. Galanina, Michael I. Bird, Nikolay E. Nifanr'ev, Nicolai V. Bovin. Assembly of P-Selectin Ligands on a Polymeric Template. Chem&Biol, Vol. 9, 2002, p. 757-762) as well as those documents cited below, synthetic manipulations of a molecules charge will influence its selectin-binding properties as much as the optmization of a carbohydrate ligand. The polyacrylamide conjugate SiaLe^{a} inhibits P-selectin with an IC₅₀ of 40 μM (by SiaLe^{a} residue) or 0.4 μM for the whole macromolecule (Tatyana V. Pochechueva, Oxana E. Galanina, Michael I. Bird, Nikolay E. Nifant'ev, Nicolai V. Bovin. Assembly of P-Selectin Ligands on a Polymeric Template. Chem&Biol, Vol. 9, 2002, p. 757-762). In comparison, sulfatide (3-0-sulfated galactocerebroside) and some of its synthetic analogues demonstrate an IC₅₀ of 0.1-1 μM (A. Marinier, A. Martel, J. Banville, C. Bachand, R. Remillard, R. Remillard, P. Lapointe, B. Turmel, M. Menard, W. E. Harte Jr., J. J. K. Wright, G. Todderud, K. M. Tramposch, J. Bajorath, D. Hollenbaugh, A. Aruffo. Sulfated galactocerebrosides as potential antfinflammatory agents. J. Med. Chem., 1997, 40, 3234-3247). For the highly sulfated polysaccharide fucoidan from algae, which is known to be the most potent among those described, the IC₅₀ is 0.1 μM (it is a regular polysaccharide with repeating hexasaccharide units) (Tatyana V. Pochechueva, Oxana E. Galanina, Michael I. Bird, Nikolay E. Nifarit'ev, Nicolai V. Bovin. Assembly of P-Selectin Ligands on a Polymeric Template Chem&Biol, 2002, submitted; Ley, K., Linnemann, G., Meinen, M., Stoolman, L. M., and Gaethgens, P. (1993) Fucoidin, but not yeast polyphosphomannan PPME, inhibits leukocyte rolling in venules of the rat mesentery. Blood 81, 177-185).

High-affinity binding of P-selectin with PSGL-1 requires the latter to contain both, a carbohydrate chain with SiaLe^{x} and sulfated tyrosine residues on the polypeptide chain. It was surprisingly demonstrated that an sTyr-based monoligand conjugate possesses a P-selectin binding activity that is comparable to that of PSGL-1 and fucoidan. Because the three sulfotyrosine residues in PSGL-1 appear to be adjacent, i.e. sTyr₄₆-X-sTyr₄₈-X—X-sTyr₅₁ (A. Leppanen, S. P. White, J. Helin, R. P. McEver, R. D. Cummings. Binding of glycosulfopeptides to P-selectin requires stereospecific contributions of individual tyrosine sulfate and sugar residues J. Biol. Chem, 275, 39569-39578 (2000)), those mimetics of the present invention were designed to provide a maximum or near maximum density of sulfated residues. Two low molecular weight compounds, sTyr-sTyr-sTyr (#13, Table 1) and hexa-O-sulfo-aminoglucitol (#14) were also synthesized, where the density of HSO₃ group was rather high, in particular in the second one. However, both compounds were unable to inhibit the recombinant P-selectin in the cell-free assay (Table 1, see below).

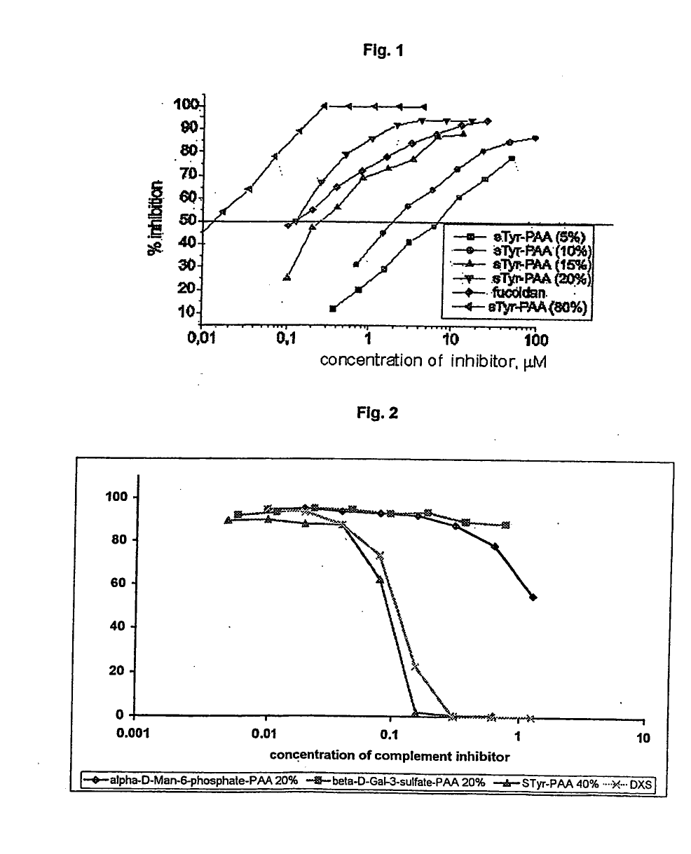

On the other side, multivalent compounds such as the PAA conjugates with a high sTyr loading display a dramatic increase in P-selectin-blocking potency (FIG. 1). A twofold sTyr loading increase in the conjugates containing sTyr (in range 5to 80 mol %) gave a tenfold higher activity. The most potent inhibitor was the 80 mol-% sTyr conjugate (higher loading is problematic but possible) which had an IC₅₀ value of 10 nM for one sTyr residue (i.e. ˜0.1 nM if counted for the whole macromolecule) or 6 ng/ml in weight. This highly sulfated conjugate was a significantly more potent inhibitor of P-selectin than PSGL-1 and fucoidan.

Of importance for a higheractivity is not only a high negative charge of the molecule, but also the origin of an anion group as well as its proximity. Three results obtained in vitro confirm this finding. First, the conjugate HSO₃Le^{a}-PAA-sTyr (15/5=the polyacrylamide containing 155 mol HSO₃Le^{a} and 5 % mol sTyr) (see substance 6 in table 1 below) with a total amount of sulfogroups being the same as for sTyr-PAA (20) appeared to be sixty times less active than the latter. Secondly, the PAA-derivative of HSO₃OCH₂CH₂— (see substance 9 in table 1 below) is less active by three orders of magnitude when compared to the corresponding derivative of sTyr (see substance 1 in table 1 below), i.e. the sTyr-PAA-P-selectin interaction is not exclusively Coulomb in nature. Instead, the aromatic tyrosine residue is important, too. Finally, Table 1 presents data for a different polyanion, being just the SiaLe^{a}-conjugate of a polyacrylic acid. According to Table 1 and FIG. 1 the derivative of a polyacrylic acid, SiaLe^{a}-pA (see substance 3 in table 1 below), containing 20 mol % oligosaccharide and 80 mol % of carboxyl groups is ten times less potent than a similar compound based on sulfotyrosine,. i.e. SiaLe^{a}-PAA-sTyr (see substance 5 in table 1 below).

Thus, it was surprisingly found that for reaching high-affinity binding to P-selectin, a polymeric molecule may lack a carbohydrate Jigand, but must contain a high content of sulfoty-rosine. The interaction stoichiometry of sTyr-PAA binding with P-selectin in the solid phase assay seems to be 1:1, indicating that the high activity is not the result of cross-binding between one molecule of sTyr-PAA with several P-selectin molecules. Although the present invention is not bound by theory, it seems that due to the high density of sTyr residues in the conjugates off the present invention these compounds bind to all of the three positively charged amino acid residues in the Tyr-binding site and due to the sufficient size of the conjugate the lectin site of P-selectin is shielded sterically. Due to the spatial proximity, it cannot be exclude that some of the sTyr residues interact with the lectin site, i.e. with the arginine which should bind to SiaLe^{x} carboxyl. The above mentioned model explains both, the need for high density of sTyr and the need for a large molecular size of the compound that promotes the blocking of simultaneous binding to the two distinct sites of P-selectin.

Table 1 shows the inhibition of HSO₃Le^{a}-PAA-biotin binding with P-selectin by monomeric and polymeric compounds

| | |
|---|---|
| INHIBITOR | IC₅₀, μM¹ |

Multimeric

| | | |
|---|---|---|
| 1. sTyr-PAA (80) | 0.01 | (6 ng/ml) |
| 2. Fucoidan | 0.1 | (100 ng/ml)² |
| 3. SiaLe^{a}-polyacrylic acid | 2 | |
| 4. SiaLe^{a}-PAA-sTyr (20/10)³ | 10 | |
| 5. SiaLe^{a}-PAA-sTyr (20/max)⁴ | 0.1 | |
| 6. HSO₃Le^{a}-PAA-sTyr (15/5) | 25 | |
| 7. SiaLe^{a}-PAA | 40 | |
| 8. HSO₃Le^{a}-PAA | 120 | |
| 9. HSO₃OCH₂CH₂-PAA (80) | 40 | (6 μg/ml) |

Monomeric

| | | |
|---|---|---|
| 10. SiaLe^{a}, SiaLe^{x} | >1500 | |
| 11. HSO₃Le^{a} | Nl | (1.5 mM) |
| 12. sTyr | Nl | |
| 13. sTyr-sTyr-sTyr | Nl | |
| 14. Hexa-O-sulfo-aminoglucitol | Nl | |

¹The Values for the inhibition were the means of at least triplicate determinations. Standard deviations were less than 10%.
²The molar concentration calculation is based on an assumption that the hexasaccharide is an active unit.
³The figure in brackets designates the mol-% of the given ligand in the polyacrylamide conjugate; two figures separated via “/” means two ligands; if no-sign it is 20 mol-%.
⁴“max” means that after introduction of 20 mol-% carbohydrate ligand to the polymer it was subjected to action of excess sTyr resulting in maximum possible content of sTyr, which in reality was about 60%.

Results of Rat Peritonitis Model

Peptone-induced acute rat peritonitis was characterized by neutrophil extravasation into the peritoneal cavities. Intravenous injection of the inhibitors resulted in different degrees of inhibition of neutrophil extravasation. Table 2 (see below) provides the results of this assay. According to the results only fucoidan and three of the tested synthetic substances according to the invention definitely blocked the inflammation process. sTyr-PAA (80 mol %) demonstrated a dramatic activity in vitro, while it turned out to be a little less active than fucoidan (Table 2) in the rat in vivo model. This result reflects that P-selectin binding can be completely blocked in vitro while in vivo there are a number of other mechanisms that promote leukocyte extravasation. Inositol hexaphosphate (O.Cecconi, R. N. Nelson, W. G. Roberts, K. Hanasaki, G. Mannori, C. Schultz, T. R. Ulich, A. Aruffo, M. P. Bevilacqua. Inositol polyanions. Noncarbohydrate inhibitors of L- and P-selectin that block inflammation. J. Biol. Chem., 269, 15060-15066 (1994)) was yet less active in a mouse test system inhibiting the yield of neutrophils (almost half of them) in the dose about 70 mg/kg. A natural ligand such as recombinant PSGL-1 acts in a dose of about 1 mg/kg (S. P. Khor, K. McCarthy, M. Dupont, K. Murray, G. Timony. Pharmacokinetics, pharmacodynamics, allometry, and dose selection of rPSGL-Ig for phase I trial. J. Pharmacol. Exp. Ther., 293, 618-624 (2000); R. Hayward, B. Campbell, Y. K. Shin, R. Scalia, A. M. Lefer. Recombinant soluble P-selectin glycoprotein ligand-1 protects against myocardial ischemic reperfusion injury in cats. Cardiovascular Res., 41, 65-76 (1999)) which approaches the fucoidan activity under study. The low activity of synthetic high molecular weight blockers of P-selectin was observed before (Natalia Ushakova, Marina Preobrazhenskaya, Mike I. Bird, Richard Priest, A. V. Semenov, A. V. Mazurov, Nikolay Nifant'ev, Tatyana Pochechueva, Oxana Galanina, Nicolai Bovin. Monomeric and Multimeric Blockers of Selectins: Comparison of In Vitro and In Vivo Activity/Biol. Chem. Hoppe Seyler, 2002 submitted). To reach an appropriate in vivo inhibition, even strong substance-selectin binding does not seem sufficient. Additional properties seem to be of importance, too, i.e. the ability of rolling as well as tropism to the corresponding target cells and at the same time a minimum binding to other cells.

Table 2 shows the degree of inhibition of rat peritonitis by sialylated and sulfated PAA-conjugates in comparison with fucoidan and free SiaLe^{x} tetrasaccharide. The data are presented as means ± SEM.

| | | | | |
|---|---|---|---|---|
| | Number | | | |
| | of rats | Number of neutrophils | Mean inhibition | Dose, mg |
| Preparation | in group | per rat × 10⁻⁶ | (% to control) | per rat |

Series 1

| | | | | |
|---|---|---|---|---|
| Control group (no preparation) | 12 | 41.4 ± 5.4 | | — |
| SiaLe^{a}-PAA-sTyr (20/10) | 8 | 19.0 ± 3.8 | 54 (p < 0.01) | 1.5-3.0 |
| SiaLe^{x} | 11 | 37.0 ± 5.6 | 11 | 1.0-3.0 |
| SiaLe^{x}-PAA | 9 | 30.0 ± 6.2 | 27 | 1.0 |

Series 2

| | | | | |
|---|---|---|---|---|
| Control group (no preparation) | 19 | 32.7 ± 2.9 | | — |
| sTyr-PAA (80) | 5 | 11.4 ± 0.6 | 65 (p < 0.001) | 2.0 |
| sTyr-PAA (80) | 5 | 23.5 ± 3.8 | 28 | 1.0 |
| Fucoidan | 10 | 2.5 ± 0.5 | 92 (p < 0.001) | 1.0 |
| HSO₃OCH₂CH₂-PAA (80) | 5 | 17.4 ± 5.8 | 47 (p < 0.05) | 2.0 |

### Example 2

### Effect of Different PAA-Conjugates as Well as DXS (Dextran Sulfate) on Complement Activation and Cytotoxicity

Effect of Different PAA-Conjugates on Activation of the Classical Complement Pathway (CH50 Test)

PAA-conjugates of several different phosphorylated or sulfated sugars and amino acids were tested for inhibition of complement activation in a standard hemolytic assay (CH50 test) with antibody-sensitized sheep erythrocytes. The tested substances were alpha-D-Man-6-phosphate-PAA (20%), beta-D-Gal-3-sulfate-PAA (20%), and sTyr-PAA (40% ). Fresh human serum was mixed with serial dilutions of the inhibitors (DXS as well as the different PAA-conjugates) at a final serum concentration of 0.5% and immediately incubated with erythrocytes for 1 h at 37° C. in a shaking water bath. After centrifugation hemolysis was determined by measuring the absorption of the supernatant at 414 nm.

Dose-response curves for the selected substances, in comparison to DXS (dextran sulfate), are given below in FIG. 2. The sTyr-PAA conjugate with a 40% substitution rate was the best complement inhibitor of all substances tested, demonstrating a stronger inhibitory activity than DXS.

Effect of DXS (Dextran Sulfate) as Well as PAA-sTyr on Complement and Coagulation

Several different variants of PAA-conjugates were tested first in a CH50 assay for complement inhibition, and good complement inhibitors were then further tested in an aPTT assay (activated partial thromboplastin time, a standard assay for coagulation) for their effect on the coagulation system.

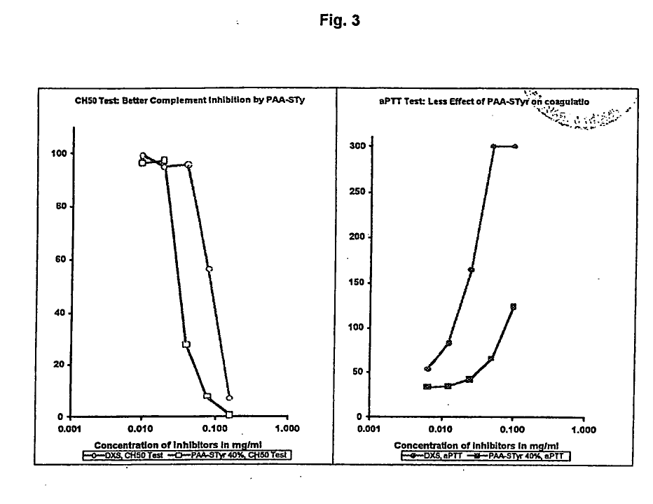

The CH50 test for evaluation of complement inhibition (see left panel of FIG. 3) was performed as described for FIG. 2. To check the effect of the substances on coagulation they were added as serial dilutions to human plasma (final concentration 50%) and the coagulation time of the mixtures was measured in a standard aPTT assay (see right panel of FIG. 3); FIG. 3 shows that DXS already leads to a prolongation of the aPTT (filled circles) to more than 300 seconds at a concentration of 0.05 mg/ml, with only a minimal inhibition of complement activation (empty circles) at that concentration. In contrast, PAA-STyr (40%) shows only a slight prolongation of the aPTT (filled squares) at 0.05mg/ml, and reduces complement activation to 27% at that concentration (empty squares). Therefore, PAA-STyr with a 40% substitution rate was both a better complement inhibitor than DXS and a much less potent anti-coagulant than the latter.

1. Polyacrylamide conjugate of the general formula I, 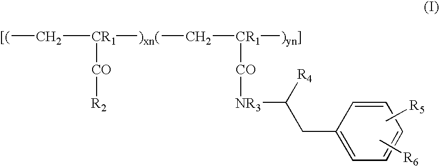
wherein
R₁ denotes hydrogen or methyl,
R₂ denotes N(R₇R₈) or OH,
R₃ denotes a hydrogen, C₁₋₆ alkyl or C₃₋₆ cycloalkyl,
R₄ denotes H or COO⁻M⁺,
R₅, R₆ denote, in each case independently of one another a hydrogen, SO₃⁻M⁺ or OSO₃⁻M⁺,
R₇, R₈ denote, in each case independently of one another, hydrogen, C₁₋₆ alcohol, C₁₋₆ alkyl, phenyl, benzyl, phenethyl or N(R₇R₈) denotes a N(CH₂)₂₋₆ ring that may also be substituted,
n is 20 to 500,
y is from 0.2 to 1.0,
x is 1 -y,
M⁺ is a physiologically acceptable monovalent cation,
and their diastereomers or enantiomers in the form of their acids or salts of physiologically compatible bases.

2. Polyacrylamide conjugate of claim 1, characterized in that R₁ denotes hydrogen.

3. Polyacrylamide conjugate of claim 1, characterized in that R₂ denotes N(R₇R₈).

4. Polyacrylamide conjugate of claim 1, characterized in that R₃ denotes hydrogen.

5. Polyacrylamide conjugate of claim 1, characterized in that R₄ denotes COO₋M⁺.

6. Polyacrylamide conjugate of claim 1, characterized in that R₆ is hydrogen and R₅ is SO₃⁻M⁺ or OSO₃⁻M⁺ in the meta or para position, preferably in the para position, most preferably R₅ is OSO₃⁻M⁺ in the para position.

7. Polyacrylamide conjugate of claim 1, characterized in that R₅ and R₆ both denote hydrogen.

8. Polyacrylamide conjugate of claim 1, characterized in that R₇ is hydrogen and R₈ is a C₁₋₆ alkohol, preferably a C₁₋₄ alkohol, most preferably ethyl alcohol.

9. Polyacrylamide conjugate of claim 1, characterized in that the counterion M⁺ is selected from the group of Na⁺, K⁺, NH₄⁺, Et₃NH⁺, HO(CH₂)NH₃⁺.

10. Polyacrylamide conjugate of claim 1, characterized in that n is 20 to 400, preferably 20 to 300, more preferably 20 to 100, most preferably about 20 to 80.

11. Polyacrylamide conjugate of claim 1, characterized in that y is 0.2 to 0.8, preferably 0.3 to 0.6, more preferably 0.3 to 0.5, most preferably 0.35 to 0.45.

12. Method for inhibiting P-selectin in vitro comprising administering the polyacrylaminde conjugate of claim 1 to a cell in a P-selectin inhibiting effective amount.

13. Method for protecting endothelial cells from complement-mediated cytotoxicity comprising administering a polyacrylamide conjugate of claim 1 to said cells in vitro.

14. A pharmaceutical composition comprising the polyacrylamide conjugate of claim 1 and a pharmaceutically acceptable carrier or excipient.

15. Method for protecting endothelial cells from complement-mediated cytotoxicity comprising administering to said endothelial cells the polyacrylamide conjugate of claim 1 in a complement-mediated cytotoxicity protecting amount.

16. Method for preventing and/or treating inflammatory reactions towards endothelial cells, preferably endothelial cells involved in arteriosclerosis or chronic heart failure comprising administering to an animal in need thereof the polyacrylamide conjugate of claim 1 in an inflammatory reactions towards endothelial cells preventing and/or treating amount.

17. Method for preventing ischemia/reperfusion damage comprising administering to an animal in need thereof the polyacrylamide conjugate of claim 1 in an ischemia/reperfusion damage preventing amount.

18. Method for the treatment of cardiac or brain infarction comprising administering to an animal in need thereof the polyacrylamide conjugate of claim 1 in a cardiac or brain infarction treating amount.

19. Method for preventing damage to organs during surgery-related ischemia comprising administering to an animal in need thereof the polyacrylamide conjugate of claim 1 in a damage to organs during surgery-related ischemia preventing amount.

20. Method for preventing acute vascular rejection reactions comprising administering to an animal in need thereof the polyacrylamide conjugate of claim 1 in an acute vascular resection reaction preventing amount.

21. Method for preventing acute a vascular rejection reaction in ABO-incompatible transplantation or xenotransplantation comprising administering to an animal in need thereof the polyacrylamide conjugate of claim 1 in an acute vascular rejection reaction preventing amount.

22. Method for safe-keeping-of life donor organs for use in transplants comprising
providing a solution comprising the polyacrylamide conjugate of claim 1, and
adding a life donor organ to said solution.

23. Method for preventing a rejection reaction during allogeneic and xenogeneic islet transplantation comprising administering to an animal in need thereof the polyacrylamide conjugate of claim 1 in a rejection reaction during allogeneic or xenogeneic islet transplantation preventing amount.

24. Method for preventing an/or treating an HIV infection comprising administering to an animal in need thereof the Polyacrylamide conjugate of claim 1 in an HIV infection preventing and/or treating amount.

25. Method for preventing and/or treating severe sepsis or septic shock comprising administering to an animal in need thereof the Polyacrylamide conjugate of claim 1 in a severe sepsis or septic shock preventing and/or treating amount.

26. Method for preventing and/or treating acute respiratory distress syndrome (ARDS) comprising administering to an animal in need thereof the polyacrylamide conjugate of claim 1 in an acute respiratory distress syndrome (ARDS) preventing and/or treating amount.
